(19) United States
(12) Patent Application Publication
(10) Pub. No.: US 2004/0044076 A1
(43) Pub. Date: Mar. 4, 2004

(54) Neutrophil imaging methods in cystic fibrosis

(75) Inventor: David Goldenberg, Mendham, NJ (US)

Correspondence Address: FOLEY AND LARDNER, SUITE 500, 3000 K STREET NW, WASHINGTON, DC, 20007, US

(73) Assignee: Immunomedics, Inc.

(21) Appl. No.: 10/452,925

(22) Filed: Jun. 3, 2003

Related U.S. Application Data
(60) Provisional application No. 60/386,411, filed on Jun. 7, 2002.

(51) Int. Cl. A61K 31/225

(52) U.S. Cl. 514/547

(57) ABSTRACT

The present invention is directed to an improved method to detect and monitor a subject having cystic fibrosis (CF) by employing at least one anti-granulocyte/neutrophil antibody or a fragment thereof and a diagnostic agent via various imaging methods, wherein said anti-granulocyte antibody is not a murine MN-3 antibody Fab′ fragment that is radiolabeled with ^{99m}Tc. Pretargeting methods for improved imaging of granulocytes accumulated in CF are also described. It is further directed to a simple, noninvasive, and effective test that can assess neutrophil delivery to the lower airways of patients with CF and other neutrophil-mediated lung diseases.

### CROSS-REFERENCE TO RELATED APPLICATIONS

This application is based on U.S. provisional patent application serial No. 60/386,411, filed Jun. 7, 2002. The entire contents of this application, including its specification, claims and drawings, are incorporated herein by reference in their entirety.

### FIELD OF THE INVENTION

The present invention is directed to an improved method to detect and monitor a subject having cystic fibrosis (CF) by employing at least one anti-granulocyte/neutrophil antibody or a fragment thereof and a diagnostic agent via various imaging methods, wherein said anti-granulocyte antibody is not a murine MN-3 monoclonal antibody Fab′ fragment that is radiolabeled with ^{99m}Tc. Pretargeting methods for improved imaging of granulocytes accumulated in CF are also described. It is further directed to a simple, noninvasive, and effective test that can assess neutrophil delivery to the lower airways of patients with CF and other neutrophil-mediated lung diseases.

### BACKGROUND OF THE INVENTION

Cystic fibrosis (CF) is the most common lethal genetic disease among Caucasians. In the United States, approximately 2,500 babies are born with CF and about 30,000 children and adults are affected by CF. CF is an autosomal recessive inherited condition that is caused by an abnormal gene on chromosome seven. The disease causes the exocrine glands of afflicted individuals to produce abnormally thick mucus that blocks passageways and produces scarring and lesions. CF affects mainly the lungs and the digestive system. In the lungs, its effects are mostly devastating; it causes increasingly severe respiratory problems. In the digestive tract, CF often results in extreme difficulty in digesting nutrients from foods.

The currently accepted pathogenic scheme for the lung disease of CF begins with a defective CF gene resulting in absent or defective protein product of the gene called Cystic Fibrosis Transmembrane Conductance Regulator (CFTR), which is involved in chloride channel activity. The primary pathophysiologic effect of this defect is believed to be the alteration of the airway environment such that abnormal mucus accounts for the airway obstruction. This is proceeded by infection with organisms with the predilection for the CF airway, such as Pseudomonas aeruginosa, Staphylococcus aureus, and Haemophilus influenzae. Besides airway obstruction by viscous secretions and chronic Pseudomonas infections as major determinants in the pathogenesis of CF, inflammation has been implicated as another major contributing factor. See Konstan, M. W. et al., Infection and Inflammation in the Lung in Cystic Fibrosis, in Cystic Fibrosis, Davis, P. B. (ed.), Marcel Dekker, Inc., NY (1993). The inflammatory response to this infection is excessive and persistent. It sets the stage for a vicious cycle of airway obstruction, infection, and inflammation that ultimately leads to lung destruction. See Davis, P. B. et al. Am. J. Respir. Crit. Care Med. 154:1229-1256 (1996) and Konstan, M. W. et al., Pediatr. Pulmonol. 24:137-142 (1997).

CFTR may affect the processing and chemical alterations of other proteins within the cell. The mechanism of this occurrence remains unclear. However, researchers have some evidence that an altered membrane protein in CF can serve as an attachment site for Pseudomonas and perhaps, aids in explaining the enhanced susceptibility of CF patients to infection.

Tissues that produce abnormal mucus secretions in CF include the airways, bile ducts of the liver, gastrointestinal tract (GIT), ducts of the pancreas, and male urogenital tract. Normal mucus forms a gel-like barrier that plays an important role in protecting the cells lining the inside surfaces of these tissues from infection. Instead of protecting these tissues from infection, abnormal mucus in CF obstructs airways and ducts, causing tissue damage. In addition, it provides an environment for bacteria to thrive. In response, the white blood cells or neutrophils (WBCs) are recruited to the lung to battle the infection. However, these cells die and release their sticky genetic material (DNA) into the mucus. This sticky DNA, in turn, aggravates the already formed abnormal mucus, causing further airway obstruction and infection.

The inflammatory component of CF is characterized by persistent infiltration of neutrophils, which includes times of clinical stability. See Konstan, M. W. et al., Am. J Respir. Crit. Care Med. 150:448-454 (1994). This occurs very early in the course of the disease for many patients, frequently during the first year of life, and may exist even in the absence of apparent infection. See Konstan, M. W. et al., Pediatr. Pulmonol. 24:137-142 (1997). In fact, bronchioalveolar lavage (BAL) studies done in the United States and Australia have found that even infants without the symptomatic lung disease developed significant endobronchial bacterial infections associated with inflammation and large numbers of neutrophils. See Khan, T. Z. et al., Am. J. Respir. Case Med. 151:1075-1082 (1995) and Armstrong, D. S. et al., BMJ 310:571-1572 (1995). In addition, inflammation was found to be present in some infants as early as 4 weeks of age in these two studies.

BAL studies also revealed that there is a severe local inflammatory response in CF patients with mild lung disease who appeared clinically healthy and free from pulmonary exacerbation. The airways of these patients contained a significant amount of bacteria, particularly Pseudomonas aeruginosa, and a marked increase of inflammatory cells and immunoglobulins (Igs). There was also a significant increase in the amount of uninhibited (active) neutrophil elastase in the epithelial lining fluid, presumably due to the excessive number of neutrophils in the airways. Active elastase has been shown to damage the lungs. See Bruce, M. C. et al., Am. Rev. Respir. Dis. 132:529-535 (1985). The elastase cleaves complement receptors and opsonic complement fragments from neutrophils and Pseudomonas aeruginosa, respectively, rendering opsono-phagocytosis ineffective and prolonging infection. See Berger, M. et al, J. Clin. Invest. 84:1302-1313 (1989) and Tosi, M. F. et al., J. Clin. Invest. 86:300-308 (1990).

Because of the deleterious effects of elastase and other inflammatory mediators in the CF airways, several therapeutic interventions aimed at decreasing inflammation or interfering with the injurious products of inflammation in the CF are undergoing investigation. These include anti-inflammatory agents, such as prednisone and ibuprofen (Auerbach, H. S. et al., Lancet 2:686-688 (1985) and Konstan, M. W., et al., J. Pediatr. 118:956-964 (1991)); anti-proteases such as exogenously administered α₁-PI and secretory leukoprotease inhibitor (McElvaney, N. G. et al., Lancet 337:392-394 (1991) and McElvaney, N. G. et al., J. Clin. Invest. 90:1296-1301 (1992)); and exogenously administered deoxyribonuclease (Hubbard, R. C. et al., N. Eng. J. Med. 326:812-815 (1992)). Moreover, diagnostic antibody systems are also undergoing investigation. See Gratz et al., Eur. J. Nucl. Med., 25(4): 386-93 (1998).

Regardless of how the inflammatory process is initiated and perpetuated, it has become clear that anti-inflammation therapy should be initiated early in life and that infection should be controlled to the maximum extent if possible. However, mechanistic studies of the disease are hampered by difficulties in monitoring its status which is currently done by analysis of bronchioalveolar lavage (BAL) fluid obtained via bronchoscopy. Not only does the cost and invasiveness of this procedure limit its use but the procedure also samples less than 5 percent of the lung. Neutrophil delivery to the oral mucosa can also be used as a surrogate marker for neutrophil delivery to the mucosa of the lower airways, but the assay is quite burdensome in requiring the subject to provide timed mouthwash specimens on 9 occasions over a 2-week period. Moreover, it is not known if this assay is a valid surrogate for what occurs in the lower airways of individuals with CF.

There exists in the field a continuing need to provide an early diagnostic/detection test for CF to monitor and decrease the spread of pulmonary infection in CF patients. There continues to exist a need to develop a simple effective test that can assess neutrophil delivery to the lower airways of CF patients. There further exist a need for an optimal CF assay that involves minimal risk to and require minimal input from the patient, and is less expensive than bronchoscopy with broncheoalveolar lavage, while not compromising the test's utility.

A potential viable route to this optimal test may be via radioimmunoscintography in which radiolabeled monoclonal antibodies, their fragments and related multivalent and/or multispecific constructs are exploited to target a specific biomolecule receptor which is then characterized by imaging (e.g., Hakki, S. et al., Clin. Orthopedics 335:275-285 (1997). This method has been applied to a number of human diseases and conditions, including osteomyelitis (Harwood, S. J. et al., Cell Biophysics. 24/25:99-107 (1994); Becker, W. et al., J. Nucl. Med. 35:1436-1443 (1994)), soft-tissue infection (Barron, B. et al., Surgery 125:288-295 (1999)), appendicitis (Barron, B., et al, ibid.), and vasculitis (Jonker, N. D. et al., J. Nucl. Med. 33:491-497 (1992)). Despite the significant diagnostic power of these monoclonal antibodies in these areas, the applicability of this technology to assess inflammatory conditions in the lung of CF patients has not been explored. Therefore, a need exists to use radiolabeled monoclonal antibodies coupled with nuclear imaging to monitor the pulmonary inflammation in CF patients.

### SUMMARY OF THE INVENTION

Accordingly, it is one object of the present invention to provide a method for diagnosing cystic fibrosis (CF), particularly the early stages of CF thereby preventing the spread of bacterial infection and at the same time, minimizing the severity of the disease.

It is also an object of the invention to provide a safe and efficacious method to assay and monitor the extent of CF in a human subject suspected of having CF or being diagnosed for CF.

It is further an object of the invention to develop a method that can assess neutrophil/granulocyte delivery to the lower airways of CF patients.

In accomplishing this and other objects, there is provided, in accordance with one aspect of the present invention, a method for diagnosing cystic fibrosis (CF) in a subject, comprises administering to said subject an effective amount for diagnosis of at least one anti-granulocyte antibody or fragment thereof and a pharmaceutically acceptable carrier, said anti-granulocyte antibody or fragment thereof binding to a diagnostic agent to form an antibody conjugate, and wherein said anti-granulocyte antibody is other than a murine MN-3 monoclonal antibody Fab′ fragment that is radiolabeled with ^{99m}Tc. However, other forms of MN-3 monoclonal antibody, either as chimeric, humanized, human or murine (if the murine is other than a murine Fab′ fragment), can be used in the present invention. In addition, a murine MN-3 monoclonal antibody Fab′ fragment, can be used in the present invention as long as it is conjugated with a radionuclide other than ^{99m}Tc.

In accordance with another aspect of the present invention, there is also provided an antibody or fragment thereof that binds to a neutrophil epitope, wherein said anti-granulocyte antibody or fragment is not a murine MN-3 monoclonal antibody Fab′ fragment that is radiolabeled with ^{99m}Tc.

In another embodiment, the diagnostic agent is conjugated to a second moiety that is recognized by at least one binding arm of a bispecific or multispecific antibody, at least one arm of which consists of an anti-granulocyte antibody or fragment thereof, whereby after the anti-granulocyte antibody/bispecific antibody targets to the CF-containing granulocytes, the second agent that binds to the non-granulocyte-binding arm of the bispecific antibody, which has the diagnostic agent attached, is administered and delivers said diagnostic agent to the sites of granulocyte binding by the anti-granulocyte antibody arm. A suitable period of time is taken between the two injections, in order to allow non-targeted antibody to be cleared from the blood and the body.

A variety of anti-granulocyte antibodies can be used in the present invention. Examples include, but are not limited to, anti-NCA-90, anti-NCA-95, MN-2, MN-3, MN-15, NP-1, NP-2, BW 250/183, and MAb 47. Antibodies can also be directed to antigens present on a single granulocyte precursor, such as anti-CD-15 and anti-CD-33. See Thakur et al., J. Nucl. Med., 37:1789-95 (1996); Ball et al., J. Immunol., 30:2937-41 (1983); PCT WO 02/12347, incorporated in their entirety herein by reference. In one embodiment, the anti-granulocyte antibody is selected from the group consisting of subhuman primate antibody, murine monoclonal antibody, chimeric antibody, humanized antibody and human antibody. In a further embodiment, the targeting agent can be a reengineered form of scFv constructs, whereby diabodies, triabodies, tetrabodies and the like, including multispecific and multivalent constructs are used. For example, a multivalent, monospecific antibody can comprise two or more antigen binding sites having affinity toward a neutrophil epitope. Another example is a multivalent, multispecific antibody comprising one or more antigen binding sites having affinity toward a neutrophil epitope and one or more hapten binding sites having affinity towards hapten molecules.

In another aspect of the present invention, there is also a provision of a neutrophil epitope targeting diagnostic conjugate comprising an antibody component that comprises an anti-granulocyte MAb or fragment thereof or an antibody fusion protein or fragment thereof that binds to the epitope, wherein the antibody component is bound to at least one diagnostic agent and wherein the anti-granulocyte Mab fragment is not a murine MN-3 antibody Fab′ fragment that is radiolabeled with ^{99m}Tc.

In yet another aspect of the invention, there is also provided, a method of delivering a diagnostic agent to a target or for detecting and monitoring a subject having CF, comprising: administering to a subject the antibody as described above-mentioned sections, waiting a sufficient amount of time for an amount of the non-binding protein to clear the subject's blood stream; and administering to said subject a carrier molecule comprising a diagnostic agent that binds to a binding site of the antibody.

In yet another embodiment of the present invention, the antibody conjugate is radiolabeled and comprises a radionuclide selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ^{52m}Mn, ⁵⁵Co, ⁷²As, ⁷⁵Br, ⁷⁶Br, ^{82m}Rb, ⁸³Sr, ¹⁹⁸Au, ²²¹At, ²¹³Bi, ²²⁴Ac, ²⁰¹Tl , or other gamma-, beta-, or positron-emitters. The radionuclides are imaged using single photon emission computed tomography (SPECT)or positron emission tomography (PET).

In a further embodiment, the diagnostic agent is selected from the group consisting of paramagnetic ions and other agents for MRI, X-ray and CT contrast agents, ultrasound enhancers and fluorescence emitters. The diagnostic agent may comprise one or more radiological contrast agents for use in imaging.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples of infections where it obviously will be useful to those skilled in the prior art.

### DETAILED DESCRIPTION OF THE INVENTION

Definitions

In the description that follows, a number of terms are used extensively. The following definitions are provided to facilitate understanding of the invention.

Unless otherwise specified, “a” or “an” means “one or more.” The term “cystic fibrosis” is defined as a lethal genetic disorder of exocrine epithelial glands of multiple organs, characterized by a wide range of symptoms including pancreatic insufficiency, dehydrated airways mucus, chronic bacterial infections of the lungs, and intestinal obstruction.

The term “anti-granulocyte antibody” refers to an antibody which recognizes an antigen which is present on one or more cell-types of the neutrophil/granulocyte/myelocyte lineage.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of a murine immunoglobulin into a human variable domain.

“Fully human antibodies” have all light and heavy variable chains of human immunoglobulin, as well as all other framework structures, with no non-human components or domains present.

The term “antibody component” includes both an entire antibody and an antibody fragment.

The term “antibody fusion protein” refers to a recombinant molecule that comprises one or more antibody components and a diagnostic agent. The fusion protein may comprise a single antibody component, a multivalent combination of different antibody components or multiple copies of the same antibody component.

A “structural gene” is a DNA sequence that is transcribed into messenger RNA (mRNA) which is then translated into a sequence of amino acids characteristic of a specific polypeptide.

A “promoter” is a DNA sequence that directs the transcription of a structural gene. Typically, a promoter is located in the 5′ region of a gene, proximal to the transcriptional start site of a structural gene. If a promoter is an inducible promoter, then the rate of transcription increases in response to an inducing agent. In contrast, the rate of transcription is not regulated by an inducing agent if the promoter is a constitutive promoter.

An “isolated DNA molecule” is a fragment of DNA that is not integrated in the genomic DNA of an organism. For example, a cloned antibody gene is a DNA fragment that has been separated from the genomic DNA of a mammalian cell. Another example of an isolated DNA molecule is a chemically-synthesized DNA molecule that is not integrated in the genomic DNA of an organism.

An “enhancer” is a DNA regulatory element that can increase the efficiency of transcription, regardless of the distance or orientation of the enhancer relative to the start site of transcription.

“Complementary DNA” (cDNA) is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand.

The term “expression” refers to the biosynthesis of a gene product. For example, in the case of a structural gene, expression involves transcription of the structural gene into mRNA and the translation of mRNA into one or more polypeptides.

A “cloning vector” is a DNA molecule, such as a plasmid, cosmid or bacteriophage, that has the capability of replicating autonomously in a host cell. Cloning vectors typically contain one or a small number of restriction endonuclease recognition sites at which foreign DNA sequences can be inserted in a determinable fashion without loss of an essential biological function of the vector, as well as a marker gene that is suitable for use in the identification and selection of cells transformed with the cloning vector. Marker genes typically include genes that provide tetracycline resistance or ampicillin resistance.

An “expression vector” is a DNA molecule comprising a gene that is expressed in a host cell. Typically, gene expression is placed under the control of certain regulatory elements, including constitutive or inducible promoters, tissue-specific regulatory elements, and enhancers. Such a gene is said to be “operably linked to” the regulatory elements.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-NCA-90 monoclonal antibody fragment binds with an epitope of NCA-90.

The term “antibody fragment” also includes any synthetic or genetically engineered protein that acts like an antibody by binding to a specific antigen to form a complex. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (“sFv proteins”), and minimal recognition units consisting of the amino acid residues that mimic the hypervariable region.

An antibody conjugate is a conjugate of an antibody component with a therapeutic or diagnostic agent. The diagnostic agent can comprise a radioactive or non-radioactive label, a radiological contrast agent (such as for magnetic resonance imaging, computed tomography, X-rays or ultrasound), and the radioactive label can be a gamma-, beta-, alpha-, Auger electron-, or positron-emitting isotope.

Description

A potential viable route to this optimal test may be via radioimmunoscintography in which radiolabeled monoclonal antibodies, their fragments and related multivalent and/or multispecific constructs are exploited to target a specific biomolecule receptor which is then characterized by imaging (e.g., Hakki, S. et al., Clin. Orthopedics 335:275-285 (1997)). This method has been applied to a number of human diseases and conditions, including osteomyclitis (Harwood, S. J. et al., Cell Biophysics. 24/25:99-107 (1994); Becker, W. et al., J. Nucl. Med. 35:1436-1443 (1994)), soft-tissue infection (Barron, B. et al., Surgery 125:288-295 (1999)), appendicitis (Barron, B., et al., ibid.), and vasculitis (Jonker, N. D. et al., J. Nucl. Med. 33:491-497 (1992)). Despite the significant diagnostic power of these monoclonal antibodies in these areas, the applicability of this technology to assess inflammatory conditions in the lung of CF patients has not been explored. Therefore, a need exists to use radiolabeled monoclonal antibodies coupled with nuclear imaging to monitor the pulmonary inflammation in CF patients.

The present invention provides improved methods for monitoring and diagnosing cystic fibrosis. The inventive methods utilize radiolabeled anti-granulocyte-specific antibodies that bind to the epitopes of the neutrophils, thereby tracking their migration via imaging.

The anti-granulocyte antibodies used in the present invention are directed to antigens associated with various cell-types of the granulocyte/neutrophil. A variety of these antibodies can be used in the present invention. In one embodiment, the inventive methods utilize anti-NCA-90 antibodies. A preferred example of such an antibody is MN-3. See Hansen et al., Cancer 71:3478-3485 (1993); Becker et al., Semin. Nucl. Med. 24(2):142-53 (1994). In another embodiment, anti-NCA-95 antibodies, anti-CD-33, or anti-CD-15 antibodies are used. See Thakur et al., J. Nucl. Med., 37:1789-95 (1996); Ball et al., J. Immunol., 30:2937-41 (1983); PCT WO 02/12347, incorporated in their entirety herein by reference. In still other embodiments, MN-2 and NP-2, which are class IIA anti-CEA antibodies, and MN-15 and NP-1, which are class I anti-CEA antibodies, are utilized. See Hansen et al., Cancer 71:3478-3485 (1993); Primus et al., Cancer Res. 43:686-692 (1983). Furthermore, MN-3, BW 250/183, and MAb 47 are utilized. Human and chimeric forms of these antibodies are preferred, and full-human and humanized versions are most preferred. Subhuman primate antibodies and murine monoclonal antibodies may also be utilized. Constructs of multispecific and/or multivalent scFv constructs are also suitable for this invention.

Production of Antibodies

Rodent monoclonal antibodies specific for granulocytes can be obtained by methods known to those skilled in the art. See generally, Kohler and Milstein, Nature 256:495 (1975); Coligan et al. (eds.), CURRENT PROTOCOLS IN IMMUNOLOGY (John Wiley & Sons, 1991). Briefly, monoclonal antibodies can be obtained by injecting mice with a composition comprising, for example, NCA-90, verifying the presence of antibody production by removing a serum sample, removing the spleen to obtain B-lymphocytes, fusing the B-lymphocytes with myeloma cells to produce hybridomas, cloning the hybridomas, selecting positive clones which produce anti-NCA-90 antibodies, culturing the clones that produce antibodies to the antigen and isolating the antibodies from the hybridoma cultures.

Monoclonal antibodies can be isolated and purified from hybridoma cultures by a variety of well-known techniques. Such isolation techniques include affinity chromatography with Protein-A Sepharose, size-exclusion chromatography, and ion-exchange chromatography. See Coligan et al. (eds.), CURRENT PROTOCOLS IN IMMUNOLOGY (John Wiley & Sons, 1991); Baines et al., pp. 79-104, METHODS IN MOLECULAR BIOLOGY (The Humana Press, Inc., 1992).

Suitable amounts of the NCA-90 antigen, also referred to as CD66c, can be obtained using standard techniques well-known in the art. For example, NCA-90 protein can be obtained from transfected cultured cells that overproduce NCA-90. Expression vectors that comprise DNA molecules encoding NCA-90 can be constructed using the published NCA-90 nucleotide sequence. See Oikawa et al., Biochem. Biophys. Res. Commun. 146:464-460 (1987); Wilson et al., J. Exp. Med. 173:137 (1991); Wilson et al., J. Immunol. 150:5013 (1993). Similarly, expression vectors for producing NCA-95 protein can be constructed using the published NCA-95 nucleotide sequence. See Berling et al., Cancer Res. 50:6534-6539 (1990).

As an illustration, DNA molecules encoding NCA-90 can be obtained by synthesizing DNA molecules using mutually priming long oligonucleotides. See Ausubel et al., (eds.), CURRENT PROTOCOLS IN MOLECULAR BIOLOGY (John Wiley & Sons, Inc., 1990); Wosnick et al., Gene 60:115 (1987); Ausubel et al. (eds.), SHORT PROTOCOLS IN MOLECULAR BIOLOGY (John Wiley & Sons, Inc., 1995). Established techniques using the polymerase chain reaction provide the ability to synthesize genes as large as 1.8 kilobases in length. Adang et al. Plant Molec. Biol. 21:1131 (1993); Bambot et al., PCR Methods and Applications 2:266 (1993); White (ed.), METHODS IN MOLECULAR BIOLOGY, pp. 263-268 (Humana Press, Inc., 1993). In a variation of this approach, an anti-NCA-90 monoclonal antibody can be obtained by fusing myeloma cells with spleen cells from mice immunized with a murine pre-B cell line stably transfected with NCA-90 cDNA using techniques well-known in the art.

A variety of anti-granulocyte antibodies can be used in the present invention. Examples include, but are not limited to, anti-NCA-90, anti-NCA-95, MN-2, MN-3, MN-15, NP-1, NP-2, BW 250/183, and MAb 4, as well as antibodies against CD15 and CD33. In one embodiment, the anti-granulocyte antibody is selected from the group consisting of subhuman primate antibody, murine monoclonal antibody, chimeric antibody, humanized antibody and human antibody. Antibodies can also be directed to antigens present on a single granulocyte precursor, such as anti-CD-15 and anti-CD-33. See Thakur et al., J. Nucl. Med., 37:1789-95 (1996); Ball et al., J. Immunol., 30:2937-41 (1983); PCT WO 02/12347, incorporated in their entirety herein by reference.

One example of a suitable murine anti-NCA-90 monoclonal antibody is an intact MN-3 monoclonal antibody. The MN-3 antibody was isolated from hybridomas derived from BALB/c mice which were immunized with partially purified carcinoembryonic antigen (CEA) derived from GW-39 human colon adenocarcinoma xenografts. See Hansen et al., Cancer 71:3478-3485 (1993). The MN-3 antibody is specific for the NCA-90 antigen, a homotypic adhesion molecule expressed on granulocytes, as well as normal colonic mucosa and colonic adenocarcinoma. See Becker et al., Semin. Nucl. Med. 24(2):142-53 (1994); Watt et al., Blood 78:63-74 (1991). An anti-NCA-90 antibody that is a murine MN-3 monoclonal antibody Fab′ fragment conjugated to ^{99m}Tc is not used in the present invention. However, other forms of MN-3 monoclonal antibody, either as chimeric, humanized, human or murine, can be used in the present invention. In addition, a murine MN-3 monoclonal antibody Fab′ fragment, can be used in the present invention as long as it is conjugated with a radionuclide other than ^{99m}Tc.

One example of a suitable murine anti-NCA-95 antibody is the BW 250/183 antibody. See Bosslet et al., Int. J. Cancer, 36:75-84 (1985); Meller et al., J. Nucl. Med. 39:1248-1253. Another useful anti-NCA-95 antibody is Mab 47. See Audette et al., Mol. Immunol. 24:1177-1186 (1987).

Another suitable antibody is the MN-2 monoclonal antibody. The MN-2 antibody was isolated from hybridomas derived from BALB/c mice which were immunized with partially purified carcinoembryonic antigen (CEA) derived from GW-39 human colon adenocarcinoma xenografts. See Hansen et al., Cancer 71:3478-3485 (1993). As a class IIA anti-CEA antibody, MN-2 can be identified readily using blocking assays well-known in the art. See U.S. Pat. No. 4,818,709, which is hereby incorporated by reference in its entirety.

Another suitable antibody is the MN-15 monoclonal antibody. The MN-15 antibody displays cross-reactivity between NCA-90 and NCA-95. MN-15 was isolated from hybridomas derived from BALB/c mice which were immunized with partially purified carcinoembryonic antigen (CEA) derived from GW-39 human colon adenocarcinoma xenografts. See Hansen et al., Cancer 71:3478-3485 (1993). As a class I anti-CEA antibody, MN-15 can be identified readily using blocking assays well-known in the art.

Still another suitable antibody is the NP-2 monoclonal antibody. The NP-2 has specificity similar to that of MN-2. NP-2 was isolated from hybridomas derived from BALB/c mice which were immunized with partially purified carcinoembryonic antigen (CEA) derived from liver metastases of human colonic adenocarcinoma according to the procedure of Krupey et al. (Immunochem. 9: 617 (1972)), as modified by Newman et al. (Cancer Res. 34:2125 (1974)). See Primus et al., Cancer Res. 43:686-92 (1983); U.S. Pat. No. 4,818,709.

Yet another suitable antibody is the NP-1 monoclonal antibody. The NP-1 has similar specificity to that of MN-15. NP-1 was isolated from hybridomas derived from BALB/c mice which were immunized with partially purified carcinoembryonic antigen (CEA) derived from liver metastases of human colonic adenocarcinoma according to the procedure of Krupey et al. (Immunochem. 9:617 (1972)), as modified by Newman et al. (Cancer Res. 34:2125 (1974)). See Primus et al., Cancer Res., 43:686-92 (1983); U.S. Pat. No. 4,818,709.

In an additional embodiment, an antibody of the present invention is a chimeric antibody in which the variable regions of a human antibody have been replaced by the variable regions of a murine antibody, e.g., rodent anti-NCA-90 antibody. A chimeric antibody as disclosed herein is a recombinant protein that contains the variable domains including the complementarity determining regions (CDRs) of an antibody derived from one species, preferably a rodent antibody, while the constant domains of the antibody molecule is derived from those of a human antibody. The advantages of chimeric antibodies include decreased immunogenicity and increased in vivo stability. Techniques for constructing chimeric antibodies are well-known to those of skill in the art. See Leung et al., Hybridoma 13:469 (1994).

In another embodiment, an antibody of the present invention is a subhuman primate antibody. General techniques for raising therapeutically useful antibodies in baboons may be found, for example, in Goldenberg et al., WO 91/11465 (1991), which is hereby incorporated by reference in its entirety, and in Losman et al., Int. J. Cancer 46:310 (1990).

In yet another embodiment, an antibody of the present invention is a “humanized” monoclonal antibody. That is, mouse complementarity determining regions are transferred from heavy and light variable chains of the mouse immunoglobulin, e.g., rodent anti-NCA-90 antibody, into a human variable domain, followed by the replacement of some human residues in the framework regions of their murine counterparts. Because non-human monoclonal antibodies can be recognized by the human host as a foreign protein, and repeated injections can lead to harmful hypersensitivity reactions, humanization of a murine antibody sequences can reduce the adverse immune response that patients may experience. For murine-based monoclonal antibodies, this is often referred to as a Human Anti-Mouse Antibody (HAMA) response. Preferably some human residues in the framework regions of the humanized antibody or fragments thereof are replaced by their murine counterparts. The constant domains of the antibody molecule is derived from those of a human antibody. General techniques for cloning murine immunoglobulin variable domains are described, for example, by the publication of Orlandi et al., Proc. Natl Acad. Sci. USA 86:3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Natl Acad. Sci. USA 89:4285 (1992), Sandhu, Crit. Rev. Biotech. 12:437 (1992) and Singer et al., J. Immun. 150:2844 (1993).

General techniques for cloning murine immunoglobulin variable domains are described, for example, by the publication of Orlandi et al., Proc. Nat'l Acad. Sci. USA 86: 3833 (1989), which is incorporated by reference in its entirety. Techniques for producing humanized MAbs are described, for example, by Carter et al., Proc. Nat'l Acad. Sci. USA 89: 4285 (1992), Singer et al., J. Immun. 150: 2844 (1992), Mountain et al. Biotechnol. Genet. Eng. Rev. 10: 1 (1992), and Coligan at pages 10.19.1-10.19.11, each of which is hereby incorporated by reference.

In general, the V_{κ} (variable light chain) and V_{H} (variable heavy chain) sequences for the antibodies can be obtained by a variety of molecular cloning procedures, such as RT-PCR, 5′-RACE, and cDNA library screening. Based on the V gene sequences, a humanized antibody can be designed and constructed as described by Leung et al. (Mol. Immunol., 32: 1413 (1995)), which is incorporated by reference. cDNA can be prepared from any known hybridoma line or transfected cell line producing a murine or chimeric antibody by general molecular cloning techniques (Sambrook et al., Molecular Cloning, A laboratory manual, 2^{nd} Ed (1989)).

Antibodies can generally be isolated from cell culture media as follows. Transfectoma cultures are adapted to serum-free medium. For production of chimeric antibody, cells are grown as a 500 ml culture in roller bottles using HSFM. Cultures are centrifuged and the supernatant filtered through a 0.2 μ membrane. The filtered medium is passed through a protein A column (1×3 cm) at a flow rate of 1 ml/min. The resin is then washed with about 10 column volumes of PBS and protein A-bound antibody is eluted from the column with 0.1 M glycine buffer (pH 3.5) containing 10 mM EDTA. Fractions of 1.0 ml are collected in tubes containing 10 μl of 3 M Tris (pH 8.6), and protein concentrations determined from the absorbence at 280/260 nm. Peak fractions are pooled, dialyzed against PBS, and the antibody concentrated, for example, with the Centricon 30 (Amicon, Beverly, Mass.). The antibody concentration is determined by ELISA, as before, and its concentration adjusted to about 1 mg/ml using PBS. Sodium azide, 0.01% (w/v), is conveniently added to the sample as preservative.

In another embodiment, an antibody of the present invention is a human monoclonal antibody. Such antibodies are obtained, for example, from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are well-known in the art. See Green et al., Nature Genet. 7:13 (1994); Lonberg et al, Nature 368:856 (1994); Taylor et al., Int. Immun. 6:579 (1994); Bruggeman et al., Curr. Opin. Biotechnol. 8:455-458 (1997). A fully human antibody also can be constructed by genetic or chromosomal transfection methods, as well as phage display technology, all of which are known in the art. See Aujame et al., Hum. Antibodies, 8:155-168 (1997). See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993).

A fully human antibody of the present invention, i.e., human MN-2, can be obtained from a transgenic non-human animal. See, e.g., Mendez et al., Nature Genetics, 15: 146-156 (1997); U.S. Pat. No. 5,633,425, which are incorporated in their entirety by reference. For example, a human antibody can be recovered from a transgenic mouse possessing human immunoglobulin loci. The mouse humoral immune system is humanized by inactivating the endogenous immunoglobulin genes and introducing human immunoglobulin loci. The human immunoglobulin loci are exceedingly complex and comprise a large number of discrete segments which together occupy almost 0.2% of the human genome. To ensure that transgenic mice are capable of producing adequate repertoires of antibodies, large portions of human heavy- and light-chain loci must be introduced into the mouse genome. This is accomplished in a stepwise process beginning with the formation of yeast artificial chromosomes (YACs) containing either human heavy- or light-chain immunoglobulin loci in germline configuration. Since each insert is approximately 1 Mb in size, YAC construction requires homologous recombination of overlapping fragments of the immunoglobulin loci. The two YACs, one containing the heavy-chain loci and one containing the light-chain loci, are introduced separately into mice via fusion of YAC-containing yeast spheroblasts with mouse embryonic stem cells. Embryonic stem cell clones are then microinjected into mouse blastocysts. Resulting chimeric males are screened for their ability to transmit the YAC through their germline and are bred with mice deficient in murine antibody production. Breeding the two transgenic strains, one containing the human heavy-chain loci and the other containing the human light-chain loci, creates progeny which produce human antibodies in response to immunization.

MAbs can be characterized by a variety of techniques that are well-known to those of skill in the art. For example, the ability of a MAb to bind to a particular antigen can be verified using an indirect immunofluorescence assay, flow cytometry analysis, or Western analysis.

Production of Antibody Fragments

The present invention contemplates the use of fragments of anti-NCA-90, anti-NCA-95, MN-2, MN-3, MN-15, NP-1, NP-2, BW 250/183, MAb 47, CD-15 MAb and CD-33 MAb antibodies. Antibody fragments which recognize specific epitopes can be generated by known techniques. The antibody fragments are antigen binding portions of an antibody, such as F(ab′)₂, Fab′, Fab, Fv, sFv and the like. Other antibody fragments include, but are not limited to: the F(ab)′₂ fragments which can be produced by pepsin digestion of the antibody molecule and the Fab′ fragments, which can be generated by reducing disulfide bridges of the F(ab)′₂ fragments. These methods are described, for example, by Goldenberg, U.S. Pat. Nos. 4,036,945 and 4,331,647 and references contained therein, which patents are incorporated herein in their entireties by reference. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4. Alternatively, Fab′ expression libraries can be constructed (Huse et al., 1989, Science, 246:1274-1281) to allow rapid and easy identification of monoclonal Fab′ fragments with the desired specificity. The present invention encompasses antibodies and antibody fragments.

A single chain Fv molecule (scFv) comprises a V_{L} domain and a V_{H} domain. The V_{L} and V_{H} domains associate to form a target binding site. These two domains are further covalently linked by a peptide linker (L). A scFv molecule is denoted as either V_{L}-L-V_{H} if the V_{L} domain is the N-terminal part of the scFv molecule, or as V_{L}-L-V_{L} if the V_{H} domain is the N-terminal part of the scFv molecule. Methods for making scFv molecules and designing suitable peptide linkers are described in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9: 132-137 (1991). These references are incorporated herein by reference.

The immunocongugates utilized in combination therapies of the present invention can comprise antibody fragments. Such antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment directly. These methods are described, for example, in U.S. Pat. Nos. 4,036,945 and 4,331,647 and the references contained therein. See also Nisonoff et al., Arch. Biochem. Biophys. 89:230 (1960); Porter, Biochem. J. 73:119 (1959); Edelman et al., METHODS IN ENZYMOLOGY, p. 422 (Academic Press, 1967), and Coligan et al. (eds.), CURRENT PROTOCOLS IN IMMUNOLOGY (John Wiley & Sons, 1991).

Other methods of cleaving antibodies, such as separation of heavy chains to form monovalent light-heavy chain fragments, further cleavage of fragments or other enzymatic, chemical or genetic techniques also may be used, so long as the fragments bind to the antigen that is recognized by the intact antibody. For example, Fv fragments comprise an association of V_{H} and V_{L} chains. This association can be noncovalent, as described in Inbar et al. Proc. Nat'l. Acad. Sci. USA 69:2659 (1972). Alternatively, the variable chains can be linked by an intermolecular disulfide bond or cross-linked by chemicals such as glutaraldehyde. See Sandhu, Crit. Rev. Biotech. 12:437 (1992).

Preferably, the Fv fragments comprise V_{H} and V_{L} chains which are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing sFvs are well-known in the art. See Whitlow et al., Methods: A Companion to Methods in Enzymology 2:97 (1991); Bird et al., Science, 242:423 (1988); U.S. Pat. No. 4,946,778; Pack et al., Bio/Technology 11:1271 (1993), and Sandhu, Crit. Rev. Biotech. 12:437 (1992).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See Larrick et al., Methods: A Companion to Methods in Enzymology 2:106 (1991); Ritter et al. (eds.), MONOCLONAL ANTIBODIES: PRODUCTION, ENGINEERING AND CLINICAL APPLICATION, p. 166-179 (Cambridge University Press, 1995); Birch et al. (eds.), MONOCLONAL ANTIBODIES: PRINCIPLES AND APPLICATIONS, p. 137-185 (Wiley-Liss, Inc., 1995).

Contemplated in the present invention are antibodies and fragments that bind with radioisotopes or other imaging agents or interact with a molecule that carries a diagnostic agent. Diagnositc agents, can include, for example, radioisotopes, enzymes, fluorescent labels, chemiluminescent labels, bioluminescent labels and paramagnetic labels. A radiolabeled diagnostic immunoconjugate may comprise a γ-emitting radioisotope, a positron-emitting (β⁺) radioisotope, an x-ray or computed tomography-enhancing contrast agent, a fluorescent-emitting compound, an MRI contrast agent, and/or an ultrasound enhancing agent. Particularly useful diagnostic radionuclides include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ^{52m}Mn, ⁵⁵Co, ⁷²As, ⁷⁵Br, ⁷⁶Br, ^{82m}Rb, ⁸³Sr, ¹⁹⁸Au, ²⁰¹Tl , or other gamma-, beta-, or positron-emitters, preferably with a decay energy in the range of 20 to 4,000 keV, more preferably in the range of 25 to 4,000 keV, and even more preferably in the range of 40 to 1,000 keV, and still more preferably in the range of 70 to 700 keV. Total decay energies of useful positron-emitting radionuclides are preferably <2,000 keV, more preferably under 1,000 keV, and most preferably <700 keV. Additional radionuclides useful as diagnostic agents utilizing gamma-ray detection include, but are not limited to: Cr-51, Co-57, Co-58, Fe-59, Se-75, Ru-97, In-114m, Yb-169, and Hg-197. Decay energies of useful gamma-ray emitting radionuclides are preferably 20-2000 keV, more preferably 60-600 keV, and most preferably 100-300 keV.

Suitable radioisotopes for the methods of the present invention include: Iodine-126, Bromine-77, Indium-113m, Ruthenium-95, Ruthenium-103, Ruthenium-105, Tellurium-121m, Tellurium-122m, Tellurium-125m, Thulium-165, Thulium-167, Thulium-168, Silver-111, Platinum-197, Palladium-109, Phosphorus-33, Scandium-47, Samarium-153, Lutetium-177, Rhodium-105, Praseodymium-142, Praseodymium-143, Terbium-161, Holmium-166, Gold-199, Cobalt-58, and Chromium-51. Preferably the radioisotope will emit in the 10-5,000 keV range, more preferably 50-1,500 keV, most preferably 50-500 keV.

Isotopes preferred for external imaging include: Iodine-123, Iodine-131, Indium-111, Gallium-67, Gallium-68, Ruthenium-97, Technetium-99m, Cobalt-57, Cobalt-58, Chromium-51, Iron-59, Selenium-75, Thallium-201, Fluorine-18, Technetium-94m and Ytterbium-169.

Isotopes most preferred for internal detection include: Iodine-125, Iodine-123, Iodine-131, Indium-111, Technetium-99m, Gallium-68, Fluorine-18 and Gallium-67, as well as certain beta-emitting isotopes used with beta-energy-detecting probes.

The method of the invention can be with magnetic resonance (MRI) imaging agents. The MRI imaging agents will contain magnetic resonance (MR) enhancing species rather than radioisotopes. In MRI, the signal generated is correlated with the relaxation times of the magnetic moments of protons in the nuclei of the hydrogen atoms of water molecules in the region to be imaged. The MRI enhancing agent acts by increasing the rate of relaxation, thereby increasing the contrast between water molecules in the region where the imaging agent accretes and water molecules elsewhere in the body. However, the effect of the agent is to decrease both T₁, and T₂, the former resulting in greater contrast while the latter results in lesser contrast. Accordingly, the phenomenon is concentration-dependent, and there is normally an optimum concentration of a paramagnetic species for maximum efficacy. This optimal concentration will vary with the particular agent used, the locus of imaging, the mode of imaging, i.e., spin-echo, saturation-recovery, inversion-recovery and/or various other strongly T₁-dependent or T₂-dependent imaging techniques, and the composition of the medium in which the agent is dissolved or suspended. These factors, and their relative importance are known in the art. See, e.g., Pykett, Scientific American 246:78 (1982); Runge et al., Am. J. Radiol. 141:1209 (1983). The MRI enhancing agent must be present in sufficient amounts to enable detection by an external camera, using magnetic field strengths which are reasonably attainable and compatible with patient safety and instrumental design. The requirements for such agents are well known in the art for those agents which have their effect upon water molecules in the medium, and are disclosed, inter alia, in Pykett, op. cit., and Runge et al., op. cit. MRI scans are stored in a computer and the images are processed.

A radiological contrast agent, when introduced into the body, makes an organ, or the surface of an organ, or materials within the lumen of an organ visible on imaging. Usually, a radiological contrast agent has a medium that is of greater radiographic density than the structure it outlines; occasionally lower densities are introduced. Radiological contrast agents that are particularly useful for magnetic resonance imaging comprise gadolinium, manganese, dysprosium, lanthanum, or iron ions. Additional agents include chromium, copper, cobalt, nickel, rhenium, europium, terbium, holmium, or neodymium. The antibodies and fragments thereof can also be conjugated to ultrasound contrast/enhancing agents. For example, the ultrasound contrast agent is a liposome that comprises a humanized Ab or fragment thereof. Also preferred, the ultrasound contrast agent is a liposome that is gas filled. Various radiopaque contrast agents can also be used with X-rays and computed tomography scanning, as described below.

The antibodies, fusion proteins, and fragments of this invention also can be labeled with paramagnetic ions for purposes of in vivo diagnosis. Paramagnetic ions suitable for the present invention include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III). Fluorescent labels include rhodamine, fluorescein and renographin. Rhodamine and fluorescein are often linked via an isothiocyanate intermediate.

Metals are also useful in diagnostic agents, including those for magnetic resonance imaging techniques. These metals include, but are not limited to: Gadolinium, manganese, iron, chromium, copper, cobalt, nickel, dysprosium, rhenium, europium, terbium, holmium and neodymium. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetctraacetic acid (EDTA), diethylenetriaminepentaacetic acid (DTPA), porphyrins, polyamines, crown ethers, bis-thiosemicarbazones, polyoximes, and like groups known to be useful for this purpose. Chelates are coupled to the peptide antigens using standard chemistries. The chelate is normally linked to the antibody by a group which enables formation of a bond to the molecule with minimal loss of immunoreactivity and minimal aggregation and/or internal cross-linking. Other, more unusual, methods and reagents for conjugating chelates to antibodies are disclosed in U.S. Pat. No. 4,824,659 to Hawthorne, entitled “Antibody Conjugates,” issued Apr. 25, 1989, the disclosure of which is incorporated herein in its entirety by reference. Particularly useful metal-chelate combinations include 2-benzyl-DTPA and its monomethyl and cyclohexyl analogs, used with diagnostic isotopes in the general energy range of 20 to 2,000 keV. The same chelates, when complexed with non-radioactive metals, such as manganese, iron and gadolinium are useful for MRI, when used along with the antibodies of the invention. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed by the invention.

Radiopaque and radiological contrast materials are used for enhancing X-rays and computed tomography, and include iodine compounds, barium compounds, gallium compounds, thallium compounds, etc. Specific compounds include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

The antibodies and their fragments of the present invention also can be labeled with a fluorescent compound. The presence of a fluorescently-labeled MAb is determined by exposing the antigen binding protein to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine. Fluorescently-labeled antigen binding proteins are particularly useful for flow cytometry analysis.

Alternatively, the antibodies and their fragments of this invention can be detectably labeled by coupling the antigen-binding protein to a chemiluminescent compound. The presence of the chemiluminescent-tagged MAb is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label the antibodies and fragments thereof the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, the antibodies and fragments of this invention can be detectably labeled by linking the antibody to an enzyme. When the antibody-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label antibody include malate dehydrogenase, staphylococcal nuclease, delta-V-steroid isomerase, yeast alcohol dehydrogenase, α-glycerophosphate dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase.

Those of skill in the art will know of other suitable labels which can be employed in accordance with the present invention. The binding of marker moieties to antibodies can be accomplished using standard techniques known to the art.

Production of Chimeric, Humanized, and Human Antibody Fusion Proteins

A simple method for producing chimeric, humanized, and human anti-granulocyte antibody fusion proteins is to mix the anti-granulocyte antibodies or fragments in the presence of glutaraldehyde. The initial Schiff base linkages can be stabilized, e.g., by borohydride reduction to secondary amines. A diiosothiocyanate or carbodlimide can be used in place of glutaraldehyde as a non-site-specific linker. The anti-granulocyte antibodies and fragments thereof of the present invention can also be used to produce two chimeric, humanized, or human MAbs, or fragments thereof, wherein at least two of the MAbs or fragments bind to at least two antigens specific to CF. For example, the MAbs can produce antigen specific diabodies, triabodies and tetrabodies, which are multivalent but monospecific. Antibody fusion proteins are expected to have a greater binding specificity than MAbs, since the fusion proteins comprise moieties that bind to at least two antigens. Thus, antibody fusion proteins are the preferred form of antigen binding protein for therapy.

The non-covalent association of two or more scFv molecules can form functional diabodies, triabodies and tetrabodies. Monospecific diabodies are homodimers of the same scFv, where each scFv comprises the V_{H} domain from the selected antibody connected by a short linker to the V_{L} domain of the same antibody. A diabody is a bivalent dimer formed by the non-covalent association of two scFvs, yielding two Fv binding sites. A triabody results from the formation of a trivalent trimer of three scFvs, yielding three binding sites, and a tetrabody is a tetravalent tetramer of four scFvs, resulting in four binding sites. Several monospecific diabodies have been made using an expression vector that contains a recombinant gene construct comprising V_{H1}-linker-V_{L1}. See Holliger et al., Proc. Natl. Acad. Sci. USA 90: 6444-6448 (1993); Atwell et al., Molecular Immunology 33: 1301-1302 (1996); Holliger et al., Nature Biotechnology 15: 632-631(1997); Helfrich et al., Int. J. Cancer 76: 232-239 (1998); Kipriyanov et al., Int. J. Cancer 77: 763-772 (1998); Holiger et al., Cancer Research 59: 2909-2916(1999)). Methods of constructing scFvs are disclosed in U.S. Pat. No. 4,946,778 (1990) and U.S. Pat. No. 5,132,405 (1992). Methods of producing multivalent, monospecific binding proteins based on scfv are disclosed in U.S. Pat. No. 5,837,242 (1998), U.S. Pat. No. 5,844,094 (1998) and WO-98/44001 (1998). The multivalent, monospecific antibody fusion protein binds to two or more of the same type of epitopes that can be situated on the same antigen or on separate antigens. The increased valency allows for additional interaction, increased affinity, and longer residence times. These antibody fusion proteins can be utilized in direct targeting systems, where the antibody fusion protein is attached to a diagnostic agent and the protein is administered directly to a patient in need thereof.

Bispecific antibodies can be made by a variety of conventional methods, e.g., disulfide cleavage and reformation of mixtures of whole IgG or, preferably F(ab′)₂ fragments, fusions of more than one hybridoma to form polyomas that produce antibodies having more than one specificity, and by genetic engineering. Bispecific antibody fusion proteins have been prepared by oxidative cleavage of Fab′ fragments resulting from reductive cleavage of different antibodies. This is advantageously carried out by mixing two different F(ab′)₂ fragments produced by pepsin digestion of two different antibodies, reductive cleavage to form a mixture of Fab′ fragments, followed by oxidative reformation of the disulfide linkages to produce a mixture of F(ab′)₂ fragments including bispecific antibody fusion proteins containing a Fab′ portion specific to each of the original epitopes. General techniques for the preparation of antibody fusion proteins may be found, for example, in Nisonoff et al., Arch Biochem. Biophys. 93: 470 (1961), Hammerling et al., J. Exp. Med. 128: 1461 (1968), and U.S. Pat. No. 4,331,647. Contemplated in the present invention is an antibody fusion protein or fragment thereof comprising at least one first MAb or fragment thereof and at least one second MAb or fragment thereof, other than the MAbs or fragments thereof of the present invention.

More selective linkage can be achieved by using a heterobifunctional linker such as maleimidehydroxysuccinimide ester. Reaction of the ester with an antibody or fragment will derivatize amine groups on the antibody or fragment, and the derivative can then be reacted with, e.g., and antibody Fab fragment having free sulfhydryl groups (or, a larger fragment or intact antibody with sulfhydryl groups appended thereto by, e.g., Traut's Reagent). Such a linker is less likely to crosslink groups in the same antibody and improves the selectivity of the linkage.

It is advantageous to link the antibodies or fragments at sites remote from the antigen binding sites. This can be accomplished by, e.g., linkage to cleaved interchain sulfydryl groups, as noted above. Another method involves reacting an antibody having an oxidized carbohydrate portion with another antibody which has at lease one free amine function. This results in an initial Schiff base (mime) linkage, which is preferably stabilized by reduction to a secondary amine, e.g., by borohydride reduction, to form the final composite. Such site-specific linkages are disclosed, for small molecules, in U.S. Pat. No. 4,671,958, and for larger addends in U.S. Pat. No. 4,699,784—incorporated by reference.

A polyspecific antibody fusion protein can be obtained by adding MAb antigen binding moieties to a bispecific chimeric, humanized or human antibody fusion protein. For example, a bispecific antibody fusion protein can be reacted with 2-iminothiolane to introduce one or more sulfhydryl groups for use in coupling the bispecific fusion protein to a third antigen MAb or fragment, using the bis-maleimide activation procedure described above. These techniques for producing antibody composites are well known to those of skill in the art. See, for example, U.S. Pat. No. 4,925,648, which is incorporated by reference in its entirety.

ScFvs with linkers greater than 12 amino acid residues in length (for example, 15- or 18-residue linkers) allow interacting between the V_{H} and V_{L} domains on the same chain and generally form a mixture of monomers, dimers (termed diabodies) and small amounts of higher mass multimers (Kortt et al., Eur. J. Biochem. (1994) 221: 151-157). ScFvs with linkers of 5 or less amino acid residues, however, prohibit intramolecular pairing of the V_{H} and V_{L} domains on the same chain, forcing pairing with V_{H} and V_{L} domains on a different chain. Linkers between 3- and 12-residues form predominantly dimers (Atwell et al., Protein Engineering (1999) 12: 597-604). With linkers between 0 and 2 residues, trimeric (termed triabodies), tetrameric (termed tetrabodies) or higher oligomeric structures of scFvs are formed; however, the exact patterns of oligomerization appear to depend on the composition as well as the orientation of the V-domains, in addition to the linker length. For example, scFvs of the anti-neuraminidase antibody NC10 formed predominantly trimers (V_{H} to V_{L} orientation) or tetramers (V_{L} to V_{H} orientation) with 0-residue linkers (Dolezal et al., Protein Engineering (2000) 13: 565-574). For scFvs constructed from NC10 with 1- and 2-residue linkers, the V_{H} to V_{L} orientation formed predominantly diabodies (Atwell et al., Protein Engineering (1999) 12: 597-604); in contrast, the V_{L} to V_{H} orientation formed a mixture of tetramers, trimers, dimers, and higher mass multimers (Dolezal et al., Protein Engineering (2000) 13: 565-574).

Contemplated in the present invention is a method of detecting and monitoring CF in a subject comprising administering to the subject an effective amount of a diagnostic conjugate comprising an anti-granulocyte MAb or fragment thereof or an antibody fusion protein or fragment thereof, wherein the MAb or fragment thereof or antibody fusion protein or fragment thereof is bound to at least one diagnostic agent and then formulated in a pharmaceutically suitable excipient. The anti-granulocyte MAb can comprise, for example, anti-NCA-90, anti-NCA-95, MN-2, MN-3, MN-15, NP-1, NP-2, BW 250/183, and MAb 47. Antibodies can also be directed to antigens present on a single granulocyte precursor, such as anti-CD-15 and anti-CD-33.

The chimeric, humanized and human antibodies to be delivered to a subject can consist of the antibody alone, immunoconjugate, fusion protein, or can comprise one or more pharmaceutically suitable excipients, one or more additional ingredients, or some combination of these. The immunoconjugate, naked antibody, fusion protein, and fragments thereof of the present invention can be formulated according to known methods to prepare pharmaceutically useful compositions, whereby the immunoconjugate or naked antibody is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof. The immunoconjugate or naked antibody of the present invention can be formulated for intravenous administration via, for example, bolus injection or continuous infusion. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In a preferred embodiment, the antibody is a monovalent, monospecific antibody or fragment thereof. In a preferred embodiment, the antibody is a multivalent, monospecific antibody or fragment thereof. These preferred antibodies and fragments are utilized for direct targeting of the epitopes of the neutrophils associated with CF. A preferred method for detecting and monitoring CF in a subject, comprises producing a conjugate comprising a monovalent, monospecific antibody or multivalent, monospecific antibody or fragment thereof and a diagnostic agent, and administering the conjugate to a patient in need thereof and subsequently detecting or monitoring the diagnostic agent with known techniques.

In a preferred embodiment, the antibody is a multivalent, multispecific antibody or fragment thereof. Also preferred is a method for detecting and monitoring CF in a subject, comprising: administering a multivalent, multispecific antibody or fragment thereof comprising one or more antigen binding sites toward a CF antigen and one or more hapten binding sites to a subject in need thereof, waiting a sufficient amount of time for an amount of the non-binding protein to clear the subject's blood stream; and then administering to the subject a carrier molecule comprising a diagnostic agent that binds to the binding site of the multivalent, multispecific antibody or fragment thereof. Detection and monitoring further require the step of detecting the bound proteins with known techniques.

Chimeric, humanized, and fully human antibodies and fragments thereof are suitable for use in diagnostic methods. Accordingly, contemplated in the present invention is a method of delivering a diagnostic agent to a target comprising (i) providing a composition that comprises an anti-granulocyte antibody and (ii) administering to a subject in need thereof the diagnostic antibody conjugate. Preferably, chimeric, humanized, and fully human antibodies and fragments thereof of the present invention are used in methods for detecting and monitoring CF. In a preferred embodiment, the antibodies bind to epitopes of the neutrophils associated with CF.

Also, the present invention provides a bispecific antibody or antibody fragment having at least one arm that is reactive against a targeted tissue and at least one other arm that is reactive against a targetable construct for carrying the diagnostic agent. The targetable construct is comprised of a carrier portion and at least 2 units of a recognizable hapten. Examples of recognizable haptens include, but are not limited to, histamine succinyl glycine (HSG) and fluorescein isothiocyanate. The targetable construct may be conjugated to a variety of agents useful for treating or identifying diseased tissue. Examples of conjugated agents were described above as diagnostic agents.

The haptens of the immunogen comprise an immunogenic recognition moiety, for example, a chemical hapten. Using a chemical hapten, preferably the HSG hapten, high specificity of the linker for the antibody is exhibited. This occurs because antibodies raised to the HSG hapten are known and can be easily incorporated into the appropriate bispecific antibody. Thus, binding of the linker with the attached hapten would be highly specific for the antibody or antibody fragment.

In another embodiment, the diagnostic agent is conjugated to a second moiety that is recognized by at least one binding arm of a bispecific or multi specific antibody, at least one arm of which consists of an anti-granulocyte antibody or fragment thereof, whereby after the anti-granulocyte antibody/bispecific antibody targets to the CF-containing granulocytes, the second agent that binds to the non-granulocyte-binding arm of the bispecific antibody, which has the diagnostic agent attached, is administered and delivers said diagnostic agent to the sites of granulocyte binding by the anti-granulocyte antibody arm. A suitable period of time is taken between the two injections, in order to allow non-targeted antibody to be cleared from the blood and the body.

Expression Vectors and Host Cells

An expression vector is a DNA molecule comprising a gene that is expressed in a host cell. Typically, gene expression is placed under the control of certain regulatory elements, including constitutive or inducible promoters, tissue-specific regulatory elements, and enhancers. Such a gene is said to be “operably linked to” the regulatory elements. A promoter is a DNA sequence that directs the transcription of a structural gene. A structural gene is a DNA sequence that is transcribed into messenger RNA (mRNA) which is then translated into a sequence of amino acids characteristic of a specific polypeptide. Typically, a promoter is located in the 5′ region of a gene, proximal to the transcriptional start site of a structural gene. If a promoter is an inducible promoter, then the rate of transcription increases in response to an inducing agent. In contrast, the rate of transcription is not regulated by an inducing agent if the promoter is a constitutive promoter. An enhancer is a DNA regulatory element that can increase the efficiency of transcription, regardless of the distance or orientation of the enhancer relative to the start site of transcription.

An isolated DNA molecule is a fragment of DNA that is not integrated in the genomic DNA of an organism. An example of an isolated DNA molecule is a chemically-synthesized DNA molecule that is not integrated in the genomic DNA of an organism. Complementary DNA (cDNA) is a single-stranded DNA molecule that is formed from an mRNA template by the enzyme reverse transcriptase. Typically, a short synthetic oligo nucleotide complementary to a portion of the mRNA is employed as a primer for the initiation of reverse transcription to generate the first stand DNA. Those skilled in the art also use the term “cDNA” to refer to a double-stranded DNA molecule consisting of such a single-stranded DNA molecule and its complementary DNA strand.

A cloning vector is a DNA molecule, such as a plasmid, cosmid, or bacteriophage, that has the capability of replicating autonomously in a host cell. Cloning vectors typically contain one or a small number of restriction endonuclease recognition sites at which foreign DNA sequences can be inserted in a determinable fashion without loss of an essential biological function of the vector, as well as a marker gene that is suitable for use in the identification and selection of cells transformed with the cloning vector. Marker genes typically include genes that provide tetracycline resistance or ampicillin resistance. A recombinant host may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell. The term expression refers to the biosynthesis of a gene product. For example, in the case of a structural gene, expression involves transcription of the structural gene into mRNA and the translation of mRNA into one or more polypeptides.

Suitable host cells include microbial or mammalian host cells. A preferred host is the human cell line, PER.C6, which was developed for production of MAbs, and other fusion proteins. Accordingly, a preferred embodiment of the present invention is a host cell comprising a DNA sequence encoding and anti-granulocyte MAb, conjugate, fusion protein or fragments thereof. PER.C6 cells (WO 97/00326) were generated by transfection of primary human embryonic retina cells, using a plasmid that contained the Adserotype 5 (Ad5) E1A- and E1B-coding sequences (Ad5 nucleotides 459-3510) under the control of the human phosphoglycerate kinase (PGK) promoter. E1A and E1B are adenovirus early gene activation protein 1A and 1B, respectively. The methods and compositions are particularly useful for generating stable expression of human recombinant proteins of interest that are modified post-translationally, e.g. by glycosylation. Several features make PER.C6 particularly useful as a host for recombinant protein production, such as PER.C6 is a fully characterized human cell line and it was developed in compliance with good laboratory practices. Moreover, PER.C6 can be grown as a suspension culture in defined serum-free medium devoid of any human- or animal-derived proteins and its growth is compatible with roller bottles, shaker flasks, spinner flasks and bioreactors with doubling times of about 35 hours. Finally, the presence of EIA causes an up regulation of expression of genes that are under the control of the CMV enhancer/promoter and the presence of E13 prevents p53-dependent apoptosis possibly enhanced through over expression of the recombinant transgene. In one embodiment, the cell is capable of producing 2 to 200-fold more recombinant protein and/or proteinaceous substance than conventional mammalian cell lines.

Chimeric, Humanized, and Human Antibodies Use for Diagnosis of CF

Contemplated in the present invention is a method of detecting and monitoring CF in a subject comprising administering to the subject an effective amount of a diagnostic conjugate comprising an anti-granulocyte MAb or fragment thereof or an antibody fusion protein or fragment thereof, wherein the MAb or fragment thereof or antibody fusion protein or fragment thereof is bound to at least one diagnostic agent and then formulated in a pharmaceutically suitable excipient. In a preferred embodiment, the antibody is a monovalent, monospecific antibody or fragment thereof. Also preferred is a method for detecting and monitoring CF in a subject, comprising: administering a multivalent, multispecific antibody or fragment thereof comprising one or more antigen binding sites toward a CF antigen and one or more hapten binding sites to a subject in need thereof, waiting a sufficient amount of time for an amount of the non-binding protein to clear the subject's blood stream; and then administering to the subject a carrier molecule comprising a diagnostic agent that binds to the binding site of the multivalent, multispecific antibody or fragment thereof. In a preferred embodiment, the antibody is a multivalent, monospecific antibody or fragment thereof. Detection and monitoring further require the step of detecting the bound proteins with known techniques.

Chimeric, humanized, and fully human antibodies and fragments thereof are suitable for use in diagnostic methods. Accordingly, contemplated in the present invention is a method of delivering a diagnostic agent to a target comprising (i) providing a composition that comprises an anti-granulocyte antibody and (ii) administering to a subject in need thereof the diagnostic antibody conjugate. Preferably, chimeric, humanized, and fully human antibodies and fragments thereof of the present invention are used in methods for detecting and monitoring CF. In a preferred embodiment, the antibodies bind to epitopes of the neutrophils associated with CF.

Any of the antibodies or antibody fusion proteins and fragments thereof of the present invention can be conjugated with one or more diagnostic agents. Generally, one diagnostic agent is attached to each antibody or antibody fragment but more than one diagnostic agent can be attached to the same antibody or antibody fragment. If the Fc region is absent (for example when the antibody used as the antibody component of the immunoconjugate is an antibody fragment), it is possible to introduce a carbohydrate moiety into the light chain variable region of a full length antibody or antibody fragment. See, for example, Leung et al., J. Immunol. 154: 5919 (1995); Hansen et al., U.S. Pat. No. 5,443,953 (1995), Leung et al., U.S. Pat. No. 6,254,868, all of which are incorporated in their entirety by reference. The engineered carbohydrate moiety is used to attach the therapeutic or diagnostic agent.

Methods for conjugating peptides to antibody components via an antibody carbohydrate moiety are well known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, all of which are incorporated in their entirety by reference. The general method involves reacting an antibody component having an oxidized carbohydrate portion with a carrier polymer that has at least one free amine function and that is loaded with a plurality of peptide. This reaction results in an initial Schiff base (imine) linkage, which can be stabilized by reduction to a secondary amine to form the final conjugate.

The antibody fusion proteins and fragments thereof of the present invention comprise two or more antibodies or fragments thereof and each of the antibodies that compose this fusion protein can contain a therapeutic agent or diagnostic agent. Additionally, one or more of the antibodies of the antibody fusion protein can have more than one diagnostic agent attached. Diagnostic agents can be attached to reduced SH groups and to the carbohydrate side chains.

The antibody with the diagnostic agent may be provided as a kit for human or mammalian diagnostic use in a pharmaceutically acceptable injection vehicle, preferably phosphate-buffered saline (PBS) at physiological pH and concentration. The preparation preferably will be sterile, especially if it is intended for use in humans. Optional components of such kits include stabilizers, buffers, labeling reagents, radioisotopes, paramagnetic compounds, second antibody for enhanced clearance, and conventional syringes, columns, vials and the like.

Administration of antibodies, antibody components, or fusion proteins to a patient can be intravenous, intraarterial, intraperitoneal, intramuscular, subcutaneous, intrapleural, and intrathecal. When administering by injection, the administration may be by continuous infusion or by single or multiple boluses.

In general, the dosage of administered anti-granulocyte antibodies will vary depending upon such factors as the patient's age, weight, height, sex, general medical condition, disease state and previous medical history. Typically, it is desirable to provide the recipient with a dosage of antibody which is in the range of from about 1 pg/kg to 20 mg/kg (amount of agent/body weight of patient), although a lower or higher dosage also may be administered as circumstances dictate. The label selected may also affect the dosage requirements.

### EXAMPLES

The embodiments of the invention may be further illustrated through examples which show aspects of the invention in detail. These examples illustrate specific elements of the invention and are not to be construed as limiting the scope of the claims.

### Example 1

A 14-year-old boy has an exacerbation of his CF lung disease, and shows evidence of infection because of high fever, malaise, and a leukocytosis. His physician decides to evaluate him with an antigranulocyte imaging method involving pretargeting. The bispecific antibody composed of MN-3 Fab′ and Mab 732 anti-In-DTPA Fab′ is injected at a dose of 5 mg. Forty-eight hours later, 131-I-labeled In-DTPA peptide is administered with 7 mCi I-131, and the patient receives gamma imaging of the chest 4 and 24 hrs later. The scans show expansive infiltration of both lungs, particularly in the upper, central regions, estimated to be about 5-fold more activity than normal. Collateral CT images assists in quantification of the scans, supporting the extensive lung involvement of activated granulocytes and the institution of aggressive antibiotic therapy.

### Example 2

A 10-year-old boy with known CF lung disease that appears to be progressing despite therapy is injected i.v. with 111-indium-labeled humanized MN-2 monoclonal antibody at a protein dose of 1 mg conjugated with 5 mg of In-111 by a DOTA chelate. Forty-eight hours later, planar and SPECT scans of the chest reveal considerable diffuse radioactivity, consistent with a pulmonary exudates or infiltration. The patient is then given high-dose antibiotic chemotherapy for 2 weeks, and shows some relief and improved pulmonary function. A repeat of the radioimmunoscintigraphy study at 4 weeks post chemotherapy reveals improvement of the lungs by a reduction of the radioactive infiltrate by approximately fifty percent.

It will be apparent to those skilled in the art that various modifications and variations can be made to the products, compositions, methods and processes of this invention. Thus, it is intended that the present invention cover such modifications and variations, provided they come within the scope of the appended claims and their equivalents.

The disclosure of all publications cited above are expressly incorporated herein by reference in their entireties to the same extent as if each were incorporated by reference individually.

We claim:

1. A method for detecting and monitoring cystic fibrosis (CF) in a subject, comprising administering to said subject, an effective amount for diagnosis, at least one anti-granulocyte antibody or fragment thereof and a pharmaceutically acceptable carrier, said anti-granulocyte antibody or fragment thereof binds to a diagnostic agent to form an antibody conjugate, wherein said anti-granulocyte antibody is not a murine MN-3 antibody Fab′ fragment that is radiolabeled with ^{99m}Tc.

2. The method of claim 1, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD15, and anti-CD-33 antibodies.

3. The method of claim 1, wherein said anti-granulocyte antibody is selected from the group consisting of murine monoclonal antibody, subhuman primate antibody, chimeric antibody, humanized antibody, and human antibody.

4. The method of claim 1, wherein said diagnostic agent is a radioactive label with an energy between 20 and 4,000 keV.

5. The method of claim 4, wherein said radioactive label is a gamma-, beta- or a positron-emitting isotope.

6. The method of claim 5, wherein said radioactive label is selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ^{52m}Mn, ⁵⁵Co, ⁷²As, ⁷⁵Br, ⁷⁶Br, ^{82m}Rb, ⁸³Sr, ¹⁹⁸Au, ²⁰¹Tl , or other gamma-, beta-, or positron-emitters.

7. The method of claim 6, wherein said radioactive labels are imaged using computed tomography (CT), single photon emission computed tomography (SPECT), or positron emission tomography (PET).

8. The method of claim 1, wherein said diagnostic agent is a radiological contrast agent.

9. The method of claim 8, wherein said radiological contrast agent is a paramagnetic ion.

10. The method of claim 9, wherein said paramagnetic ion is a metal comprising chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

11. The method of claim 8, wherein said radiological contrast agent is selected from the group comprising lanthanum (III), gold (III), lead (II), bismuth (III), nickel, rhenium, and europium.

12. The method of claim 1, wherein said diagnostic agent is a radiopaque compound selected from the group comprising iodine compounds, barium compounds, gallium compounds, thallium compounds.

13. The method of claim 12, wherein said radiopaque compound is selected from the group comprising barium, diatrizoate, ethiodized oil, gallium citrate, iocarmic acid, iocetamic acid, iodamide, lodipamide, iodoxamic acid, iogulamide, iohexol, lopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

14. The method of claim 8, wherein said radiological contrast agent is an ultrasound enhancing agent.

15. The method of claim 14, wherein said ultrasound enhancing agent is a liposome that comprises an anti-granulocyte antibody or fragment thereof.

16. The method of claim 15, wherein said anti-granulocyte antibody is selected from the group consisting of murine monoclonal antibody, subhuman primate antibody, chimeric antibody, humanized antibody, and human antibody.

17. The method of claim 15, wherein said liposome is gas filled.

18. An antibody or fragment thereof that binds to a neutrophil epitope, wherein said antibody fragment is not a murine MN-3 antibody Fab′ fragment that is radiolabeled with ^{99m}Tc.

19. The antibody or fragment thereof of claim 18, wherein said antibody or fragment thereof is an anti-granulocyte antibody or fragment thereof.

20. The antibody or fragment thereof of claim 19, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

21. The antibody or fragment thereof of claim 20, wherein said antibody or fragment thereof is chimeric.

22. The antibody or fragment thereof of claim 20, wherein said antibody or fragment thereof is humanized.

23. The antibody or fragment thereof of claim 20, wherein said antibody or fragment thereof is fully human.

24. A multivalent, monospecific antibody comprising two or more antigen binding sites having affinity toward a neutrophil epitope.

25. The multivalent, monospecific antibody of claim 24, wherein said antigen binding sites comprise an anti-granulocyte antibody or fragment thereof.

26. The multivalent, monospecific antibody of claim 25, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

27. The multivalent, monospecific antibody of claim 25, wherein said antibody or fragment thereof is chimeric.

28. The multivalent, monospecific antibody of claim 25, wherein said antibody or fragment thereof is humanized.

29. The multivalent, monospecific antibody of claim 25, wherein said antibody or fragment thereof is fully human.

30. A multivalent, multispecific antibody comprising one or more antigen binding sites having affinity toward a neutrophil epitope and one or more hapten binding sites having affinity towards hapten molecules.

31. The antibody of claim 30, wherein said antibody is chimerized.

32. The antibody of claim 30, wherein said antibody is humanized.

33. The antibody of claim 30, wherein said antibody is a human antibody.

34. A neutrophil epitope targeting diagnostic conjugate comprising an antibody component comprising an anti-granulocyte MAb or fragment thereof or an antibody fusion protein or fragment thereof of any one of claims 18-33 that binds to said epitope, wherein said antibody component is bound to at least one diagnostic agent and wherein said anti-granulocyte Mab fragment is not a murine MN-3 antibody Fab′ fragment that is radiolabeled with ^{99m}Tc.

35. The diagnostic conjugate of claim 34, wherein said diagnostic agent is a radioactive label with an energy between 20 and 4,000 keV.

36. The diagnostic conjugate of claim 35, wherein said radioactive label is a gamma-, beta- or a positron-emitting isotope.

37. The diagnostic conjugate of claim 36, wherein said radioactive label is selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn ^{52m}Mn, ⁵⁵Co, ⁷²As, ⁷⁵Br, ⁷⁶Br, ^{82m}Rb, ⁸³Sr, ¹⁹⁸Au, ²⁰¹Tl , or other gamma-, beta-, or positron-emitters.

38. The diagnostic conjugate of claim 34, wherein said diagnostic agent is a radiological contrast agent.

39. The diagnostic conjugate of claim 38, wherein said radiological contrast agent is a paramagnetic ion.

40. The diagnostic conjugate of claim 39, wherein said paramagnetic ion is a metal selected from the group comprising chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

41. The diagnostic conjugate of claim 38, wherein said radiological contrast agent is a metal selected from the group comprising lanthanum (III), gold (III), lead (II), bismuth (III), nickel, rhenium, and europium.

42. The diagnostic conjugate of claim 34, wherein said diagnostic agent is a radiopaque compound selected from the group comprising iodine compounds, barium compounds, gallium compounds, thallium compounds.

43. The diagnostic conjugate of claim 42, wherein said radiopaque compound is selected from the group comprising barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, lothalamic acid, iotroxic acid, ioxaglic acid, ioxotrizoic acid, ipodate, meglumine, metrizamide, metrizoate, propyliodone, and thallous chloride.

44. The diagnostic conjugate of claim 38, wherein said radiological contrast agent is an ultrasound enhancing agent.

45. The diagnostic conjugate of claim 44, wherein said ultrasound enhancing agent is a liposome that comprises an anti-granulocyte antibody or fragment thereof.

46. The antibody or fragment thereof of claim 45, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

47. The diagnostic conjugate of claim 44, wherein said liposome is gas filled.

48. An antibody fusion protein or fragment thereof comprising at least two anti-granulocyte MAbs or fragments thereof, wherein said MAbs or fragments thereof are selected from said the MAb or fragment thereof of any one of claims 18-47.

49. An expression vector comprising the DNA sequence of claim 48.

50. A host cell comprising the DNA sequence of claim 49.

51. A method of delivering a diagnostic agent to a target, comprising: administering to a subject the antibody of claim 30, waiting a sufficient amount of time for an amount of the non-binding protein to clear the subject's blood stream; and administering to said subject a carrier molecule comprising a diagnostic agent that binds to a binding site of said antibody.

52. The method of claim 51, wherein said carrier molecule binds to more than one binding site of the binding protein.

53. The method of claim 51, wherein said diagnostic agent is selected from the group comprising radionuclides, radiological contrast agents, and metals.

54. A method for detecting and monitoring a subject having CF, comprising: administering to a subject in need thereof the antibody of claim 30, waiting a sufficient amount of time for an amount of the non-binding protein to clear the subject's blood stream; and administering to said subject a carrier molecule comprising a diagnostic agent that binds to a binding site of said antibody.

55. The method of claim 54, wherein said diagnostic agent is selected from the group consisting of an isotope, metal, radiological contrast agent, enzyme and detecting agent.

56. The method of claim 55, wherein said diagnostic agent is an isotope.

57. The method of claim 56, wherein said isotope has a range of energy between 20 to 4,000 keV.

58. The method of claim 57, wherein said isotopes are selected from the group comprising ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ^{52m}Mn, ⁵⁵Co, ⁷²As, ⁷⁵Br, ⁷⁶Br, ^{82m}Rb, ⁸³Sr, ¹⁹⁸Au, ¹⁰²Tl , or other gamma-, beta-, or positron-emitters.

59. The method of claim 55, wherein said diagnostic agent is a metal.

60. The method of claim 59, wherein said metal is a paramagnetic ion used for MRI.

61. The method of claim 55, wherein said radiological contrast agentis chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

62. The method of claim 55, wherein said metal is selected from the group comprising lanthanum (III), gold (III), lead (II), bismuth (III), nickel, rhenium, and europium.

63. The method of claim 54, wherein said diagnostic agent is a radiopaque compound selected from the group comprising iodine compounds, barium compounds, gallium compounds, thallium compounds.

64. The method of claim 63, wherein said radiopaque compound is selected from the group comprising barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

65. The method of claim 55, wherein said detection agents are selected from the group consisting of a fluorescent compound, chemiluminescent compound, bioluminescent compound.

66. The method of claim 65, wherein said fluorescent compound is selected from the group consisting of fluorescein isothiocyanate, rhodamine, phycoerytherin, phycocyanin, allophycocyanin, o-phthaldehyde and fluorescamine.

67. The method of claim 65, wherein said chemiluminescent compound is selected from the group consisting of luminol, isoluminol, an aromatic acridinium ester, an imidazole, an acridinium salt and an oxalate ester.

68. The method of claim 65, wherein said bioluminescent compound is selected from the group consisting of luciferin, luciferase and aequorin.

69. The method of claim 55, wherein said radiological contrast agent is an ultrasound contrast agent.

70. The method of claim 69, wherein said ultrasound radiological contrast agent is a liposome that comprises an anti-granulocyte antibody or fragment thereof.

71. The antibody or fragment thereof of claim 70, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

72. The method of claim 70, wherein said liposome is gas filled.

73. A method of delivering a diagnostic agent to a target comprising (i) providing a composition that comprises an anti-granulocyte antibody and (ii) administering to a subject in need thereof the diagnostic conjugate of claim 34.

74. The method of claim 73, wherein said diagnostic agent is selected from the group consisting of an isotope, metal, radiological contrast agent, enzyme and detecting agent.

75. The method of claim 74, wherein said diagnostic agent is an isotope.

76. The method of claim 75, wherein said isotope has a range of energy between 20 to 4,000 keV.

77. The method of claim 76, wherein said isotopes are selected from the group comprising ¹¹⁰In, ¹¹¹I, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ⁵⁵Co, ⁷²As, ⁷⁵Br, ⁷⁶Br, ^{82m}Rb, ⁸³Sr, ¹⁹⁸Au, ²⁰¹T1, or other gamma-, beta-, or other positron-emitters.

78. The method of claim 74, wherein said diagnostic agent is a metal.

79. The method of claim 78, wherein said metal is a paramagnetic ion used for MRI.

80. The method of claim 74, wherein said radiological contrast agent is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

81. The method of claim 74, wherein said metal is selected from the group comprising lanthanum (III), gold (III), lead (II), bismuth (III), nickel, rhenium, and europium.

82. The method of claim 73, wherein said diagnostic agent is a radiopaque compound selected from the group comprising iodine compounds, barium compounds, gallium compounds, thallium compounds. 1

83. The method of claim 82, wherein said radiopaque compound is selected from the group comprising barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

84. The method of claim 74, wherein said detection agents are selected from the group consisting of a fluorescent compound, chemiluminescent compound, bioluminescent compound.

85. The method of claim 84, wherein said fluorescent compound is selected from the group consisting of fluorescein isothiocyanate, rhodamine, phycoerytherin, phycocyanin, allophycocyanin, o-phthaldehyde and fluorescamine.

86. The method of claim 84, wherein said chemiluminescent compound is selected from the group consisting of luminol, isoluminol, an aromatic acridinium ester, an imidazole, an acridinium salt and an oxalate ester.

87. The method of claim 84, wherein said bioluminescent compound is selected from the group consisting of luciferin, luciferase and aequorin.

88. The method of claim 74, wherein said radiological contrast agent is an ultrasound contrast agent.

89. The method of claim 88, wherein said ultrasound radiological contrast agent is a liposome that comprises an anti-granulocyte antibody or fragment thereof.

90. The antibody or fragment thereof of claim 89, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

91. The method of claim 89, wherein said liposome is gas filled.

92. A method for detecting and monitoring a subject having CF, comprising: administering to a subject in need thereof an antibody comprising at least one binding arm that has affinity to the granulocyte and at least one binding arm that has affinity to the non-granulocyte-binding protein; waiting a sufficient amount of time for an amount of the non-binding protein to clear the subject's blood stream; and administering to said subject a carrier molecule comprising a diagnostic agent that binds to a binding site of said antibody.

93. The method of claim 92, wherein said carrier molecule is selected from the group comprising HSG, fluorescein isothiocyanate, ethylenediaminetetraacetic acid (EDTA), diethylenetriaminepentaacetic acid (DTPA), porphyrins, polyamines, crown ethers, bisthiosemicarbazones, polyoximes, and other chelates.

94. The method of claim 92, wherein said diagnostic agent is selected from the group consisting of an isotope, metal, radiological contrast agent, enzyme and detecting agent.

95. The method of claim 94, wherein said diagnostic agent is an isotope.

96. The method of claim 95, wherein said isotope has a range of energy between 20 to 4,000 keV.

97. The method of claim 96, wherein said isotopes are selected from the group comprising ¹¹⁰In, ¹¹¹In, ¹⁷⁷Lu, ¹⁸F, ⁵²Fe, ⁶²Cu, ⁶⁴Cu, ⁶⁷Ga, ⁶⁸Ga, ⁸⁶Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ⁵¹Mn, ^{52m}Mn, ⁵⁵Co, ⁷²As, ⁷⁵Br, ⁷⁶Br, ^{82m}Rb, ⁸³Sr, ¹⁹⁸Au, ²⁰¹Tl, or other gamma-, beta-, or positron-emitters.

98. The method of claim 93, wherein said diagnostic agent is a metal.

99. The method of claim 98, wherein said metal is a paramagnetic ion used for MRI.

100. The method of claim 94, wherein said radiological contrast agent is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

101. The method of claim 94, wherein said metal is selected from the group comprising lanthanum (III), gold (III), lead (II), bismuth (III), nickel, rhenium, and europium.

102. The method of claim 92, wherein said diagnostic agent is a radiopaque compound selected from the group comprising iodine compounds, barium compounds, gallium compounds, thallium compounds.

103. The method of claim 102, wherein said radiopaque compound is selected from the group comprising barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

104. The method of claim 94, wherein said detection agents are selected from the group consisting of a fluorescent compound, chemiluminescent compound, bioluminescent compound.

105. The method of claim 104, wherein said fluorescent compound is selected from the group consisting of fluorescein isothiocyanate, rhodamine, phycoerytherin, phycocyanin, allophycocyanin, o-phthaldehyde and fluorescamine.

106. The method of claim 104, wherein said chemiluminescent compound is selected from the group consisting of luminol, isoluminol, an aromatic acridinium ester, an imidazole, an acridinium salt and an oxalate ester.

107. The method of claim 104, wherein said bioluminescent compound is selected from the group consisting of luciferin, luciferase and aequorin.

108. The method of claim 94, wherein said radiological contrast agent is an ultrasound contrast agent.

109. The method of claim 108, wherein said ultrasound radiological contrast agent is a liposome that comprises an anti-granulocyte antibody or fragment thereof.

110. The antibody or fragment thereof of claim 109, wherein said anti-granulocyte antibody or fragment thereof is selected from the group comprising anti-NCA-90, anti-NCA-95, MN-2, MN-3, MN-15, NP-1, NP-2, BW 250/183, MAb 47, anti-CD-15, and anti-CD-33 antibodies.

111. The method of claim 109, wherein said liposome is gas filled.
